(19) United States
(12) Patent Application Publication
(10) Pub. No.: US 2007/0128286 A1
(43) Pub. Date: Jun. 7, 2007

(54) Production of Polymer Matrix for Delivery (PMD)

(75) Inventors: Yawo-Kuo Twu, Taipei County (TW); Min-Wen Shieh, Taipei County (TW)

Correspondence Address: WPAT, PC;INTELLECTUAL PROPERTY ATTORNEYS, 2030 MAIN STREET, SUITE 1300, IRVINE, CA, 92614, US

(73) Assignees: A.C. (USA) INC., City of Industry, CA (US); DNAER BIOLOGICAL PRODUCTS DEVELOPMENT CO., LTD., Taipei County (TW)

(21) Appl. No.: 11/565,418

(22) Filed: Nov. 30, 2006

(30) Foreign Application Priority Data
Dec. 2, 2005 (TW) 094142402

(51) Int. Cl. A61K 9/14; C08B 37/08

(52) U.S. Cl. 424/488; 536/20; 977/906

(57) ABSTRACT

This invention is a means of production of polymer matrix for delivery. This invention employs amino acid chelated minerals as an ionic cross-linking agent, which forms colloidal solution containing delivery molecules after fully mingling with chitosan-acid solution that contains delivery molecules. This invention uses amino acid chelated minerals and chitosan to produce polymer matrix and then deliver molecules while proceeding ionic cross-linking. This is an innovative procedure.

### TECHNICAL FIELD OF THE INVENTION

This is a means of production of polymer matrix for delivery, which refers specifically to a production or product that manufactures colloidal solid structure by employing amino acid chelation and physical power. This product adopts natural biological polymer chitosan as the matrix. Chitosan's special functional group, which has bio-compatibility, bio-degradability, and can be used as the matrix of delivery, contributes to the production of the aforementioned product. This technique is under the technical field of biological material delivery.

### PRIOR ART

Bio-technology and medicine are technologies having the greatest potential in the 21^{st} century, whereas the technique of controlling material delivery has long been an active field in medical research science, which includes many scientific techniques that may improve human health. The material delivery system usually uses liposome or polymer as carriers. Recently, much attention has been put on the employment of bio-degradability to produce carrier, which is considered to be a material delivery system that has great potential.

One of the material delivery system is the Liposomal Drug Delivery System. Liposome is a bi-layer fat globule formed by lipid hydration. Due to its bi-layer structure that similar to membrane, liposome can be used to encapsulate water-affinity and water-repellent drugs, and be used as a drug carrier for the target. That liposome being used as a drug delivery system has the following features: the feature of encapsulating water-affinity and water-repellent drugs, the feature of bio-compatibility and bio-degradability, the capability of protecting drug from being degraded by metabolism after it enters the body, and the capability of having the effects of mitigation and control.

There is a variety of polymer that may be used in the drug delivery system. The polymer, which can be divided into biodegradable and non-biodegradable, includes natural polymer like collagen, polysaccharide bio-polymer like chitosan, hyaluronic acid, such chemical synthetic as polyurethane, acrylic-based polymers, and bio-absorption [symbol] poly (glycolide co-lactide), PLGA [symbol] . Generally speaking, bio-polymer, which may be absorbed or metabolized, is a more favorable option. Now, the advanced technique mainly focused on the development of the delivery system of biodegradable drug. There are two main mechanisms of decomposition: one is degradation, and the other one is erosion. Degradation is a chemical reaction that resulted from the break of chemical chains, while erosion is a physical phenomenon, which depends mainly on the speed of dissolution and expansion. To most of the biomedicine polymers, these two possibilities may happen subject to the chemical structures. Furthermore, there are many considerations as to the drug delivery system in coordination with the limitation of drug applications. If we want the drug continue to release for a period of time in the stomach of a patient, the PH value differentiation and the change of environment should be taken into account. Therefore, the design of a polymer carrier becomes very important.

This invention technique uses natural material for production. The natural polymer chitin used in this invention exists widely in low-end algae, mushroom, cell walls of fungi, stratum corneum of animals, shells of oceanic invertebrates and insects, among them shells of shrimps and crabs are the most common source of chitin. The natural polymer chitin is one of the polysaccharide that widely spread in the nature, second only to cellulose. In general, after processing wasted shells of shrimps and crabs with acid and alkaloid, and separating and extracting, we can obtain crude chitin polymer, which is academically called chitin. It is a polymeric polysaccharide formed by combining N-acetyl-D-glucosamine and β-1,4 chain, and its molecule size is decided by the source material and the subsequent processes. After chitin is processed with hot alkaloid, the N-acetyl group will be removed (mostly 70%˜90%), and then we can obtain Chitosan. The only difference between chitin and chitosan is the functional group connecting the second carbon. Chitin is -NHCOOH₃, while chitosan is -NH₂.

Chitin and chitosan, which are all bio-polymer obtained from natural biological organism, have good bio-compatibility, bio-adhesion, bio-activity, biodegradability, non-toxicity, anti-bacteria with other biological organism, and are widely applied in food, drug, chemistry, environmental protection and bio-technological fields. The reasons of chitosan being widely used in biomedical material are the following features that chitosan is a natural polymer, it has good bio-compatibility with body cells that no rejection would occur, it carries positive electric charge, and it has an active functional group. While chitosan is applied in carriers, the above features can enhance bioavailability, stabilize compounds of the substance, activate absorption, control the rate of dissolution, and help deliver the substance to the target organs.

The main purpose of this invention is to enhance the use value of chitosan by way of ionic cross-linking chitosan. Because the amino group of the second carbon of chitosan molecules tends to connect with hydrogen ion and becomes ionized, it can retain chitosan's excellent characteristics and improve its defect of low water solubility after its functional group connects with salt. Therefore, chitosan has a better property than chitin, which makes it more favorable in biomedicine applications.

### DESCRIPTION OF THE INVENTION

This invention is a means of production of polymer matrix for delivery, which in the first place mixes chitosan evenly with delivery molecules, and then speedily homogeneously mixes with amino acid chelated minerals solution to produce polymer matrix with delivery function. This invention uses natural polymer and amino acid and employs chelation and physical power to produce colloidal solid. This invention is different from other means of production using general substances for matrix delivery. Features of the recipe, production and product are as follows:

1. This invention uses chitin polymer—chitosan. The bio-polymer produced from natural biological organism has good bio-compatibility, bio-adhesion, bio-activity, bio-degradability with other biological organism, and non-toxicity, anti-bacteria, and is widely applied in food, medicine, chemistry, environmental protection, agriculture, and biotechnology fields.

2. This invention adopts two amino acid chelated minerals and proceeds ionic cross-linking with chitosan. This technique may, according to customers' needs, adjust the delivery rate of the substance delivered, to the utmost satisfaction of customers.

3. This invention adopts high-speed homogeneous mix method, by which the particle size of the polymer matrix with delivery function can be as tiny as nanometers. The particle size may be controlled to between 20 nm and 5 μm.

4. This research adopts chitosan and amino acid chelated minerals as the polymer matrix, which is used to encapsulate and carry short-chain peptide molecules. Because there is an excellent compatibility between carriers and substance being carried, the encapsulation rate and the control effect of delivery can be highly promoted.

5. The production of this research is simple and fast. The chitosan employed can be extracted and obtained from wasted shells of shrimps and crabs, which not only saves cost but also thinks highly of environmental protection. Furthermore, the employment of amino acid chelated minerals as the ionic cross-linking, in which there is no chemical reaction needed for molecule encapsulation, is an innovative production.

### EMBODIMENT

This invention utilizes amino acid chelated minerals and chitosan to proceed ionic cross-linking and produce polymer matrix with delivery function. The description on the production procedures of this invention is given as follows. Please refer to the flow chart of the attached drawing:

A: chitosan-acid solution

Chitosan is a biodegradable polymer without color, toxicity and taste. It has low water solubility, but it can dissolve in weak acid solution. That's why this invention uses weak acid solution to dissolve chitosan making it into the form of liquid solution.

B: delivery molecule solution

The delivery molecules used in this invention are short-chain peptide molecules, and the repeat unit number of amino acid is composed of 2˜30 molecules. Dissolve it in water to obtain liquid solution.

C: mix

This invention mixes the aforementioned delivery molecule solution with chitosan-acid solution, and uses a magnetic stirrer to evenly mix them.

D: amino acid chelated minerals solution

The amino acid chelated minerals solution used in this invention is asparte and glutamate carrying negative charge under the condition of PH=7. Mix and agitate amino acid chelated mineral solution in water, we can obtain amino acid chelated mineral solution for later use.

E: high-speed homogeneous mix

Add speedily the above amino acid chelated minerals solution into the homogeneously mixed solution of chitosan-acid solution and delivery molecule solution, and make it disperse homogeneously

F: buffer dolution

Prepare pH=7 buffer solution, and use it to adjust the above high-speed homogeneous solution until it becomes neutral.

G: polymer matrix with delivery function

After the above high-speed homogeneous solution is adjusted to be neutral, we can obtain encapsulated polymer matrix with delivery function.

In order to give the esteemed patent committee further understanding on the innovative effects of this invention, several embodiments are listed as follows. The following embodiments are given to make a general description on this invention, for which, we must proclaim in advance, it is not intended that the scope of the invention in any way be limited by the following description.

### Embodiment 1

Based on the recipe of No. 1˜2 on the recipe table (Table 1), dissolve 10 g chitosan of which N-acetyl group is 90% removed in 3% (w/v) acetic acid with fixed temperature of 70□. Mix and agitate in the speed of 50 rpm for 30 minutes till the solid is dissolved, add delivery molecules solution, and then evenly mix and agitate in the speed of 50 rpm for 1 hour. After the mixed solution is prepared, we homogeneously mix the solution again in a high rotation speed of 10500 rpm, and, in the same time, add speedily the amino acid chelated minerals solution for 5 minutes to let the amino acid which carries negative charge and chitosan which carries positive charge form ionic cross-linkage. The solution now is weak acid. We use PBS buffer solution of pH=7 to adjust it to be neutral and we obtain a colloidal solution with encapsulated polymer matrix with delivery function. The results are shown as the sample 1˜2.

### Embodiment 2

A means of embodiment 1 wherein the concentration of amino acid is changed from 5 mM to 10 mM, and the other processes are similar to embodiment 1. The products obtained are sample No. 3˜4

### Embodiment 3

Change the chitosan solvent of embodiment 1 and 2 from acetic acid to formic acid, the products obtained are sample No. 5˜8.

### Embodiment 4

A means of embodiment 1, but without the addition of amino acid chelated mineral solution; the other processes are similar to embodiment 1. The product obtained is sample 9.

### Embodiment 5

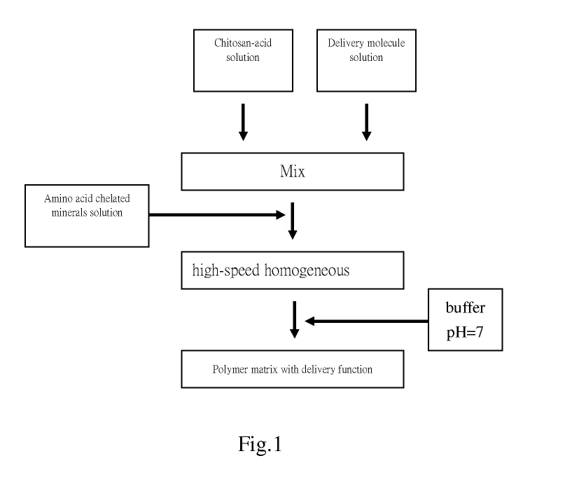

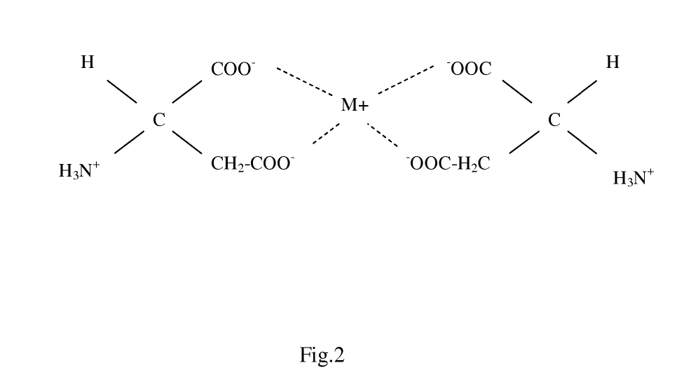

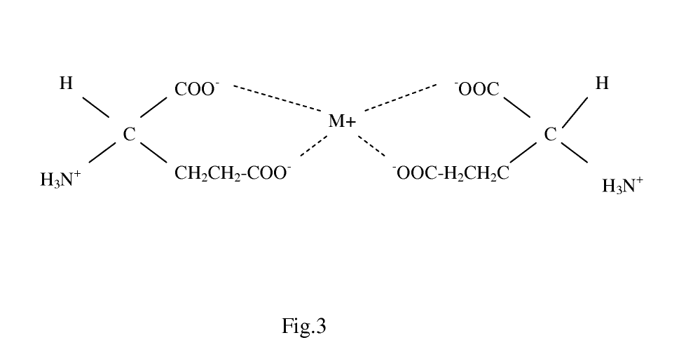

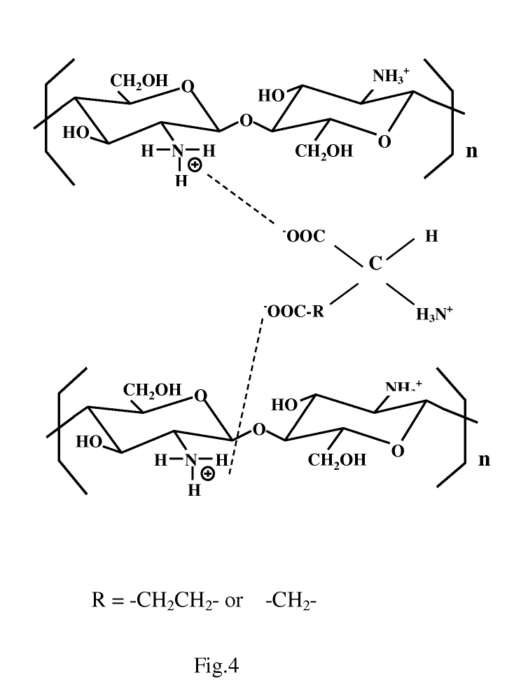

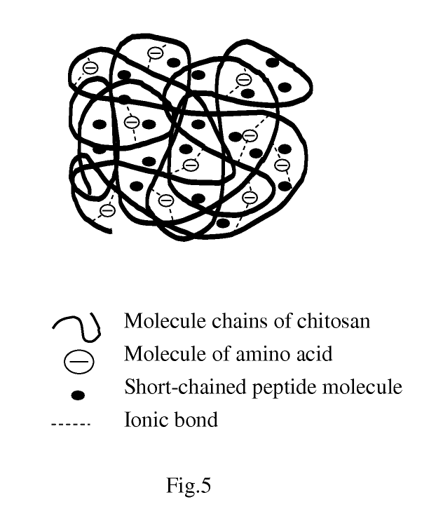

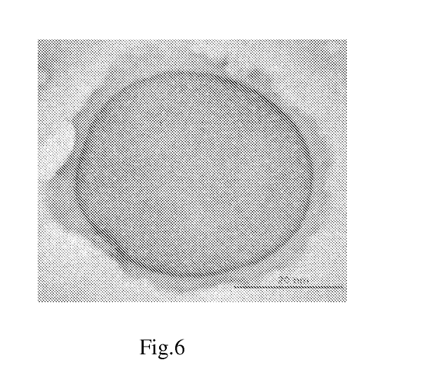

Use Transmission Electron Microscopy (TEM) to examine the shape and particle size of the above samples. The result of sample 3 is shown as FIG. 6. The particle size of the samples is shown after calculation as Table 2.

### Embodiment 6

Test the encapsulation rate of the above samples to observe how the polymer matrix with delivery function is encapsulated. First, adjust pH value to pH>9 for the above samples in order to extract chitosan carriers, and filter with 0.2 μm membrane to obtain transparent solution. Then, use High Performance Liquid Chromatography (HPLC) to measure the concentration of free thymopentin (TP-5) in the solution, and the amount of free thymopentin and the encapsulation rate can be obtained after computation. The data of the above samples measured and obtained are as Table 2. The computation formula of encapsulation rate is as follow:

$$Encapsulation rate = \frac{amount of delivered molecule used ( g ) - amount of TP \text{-} 5}{amount of delivered molecule used ( g )} \times 100 \%$$

### Embodiment 7

Test the release rate of thymopentin of the above samples to observe how the polymer matrix with delivery function is released. First, take 10 mL from each sample and add it into 90 mL PBS buffer solution, then agitate in a speed of 50 rpm and take a sample from it every 6 hours. Adjust the pH value to pH>9 for the samples taken in order to extract chitosan carriers, and filter them with 0.22 μm membrane to obtain transparent solution. Then, use High Performance Liquid Chromatography (HPLC) to measure the concentration of free thymopentin (TP-5) in the solution, and the accumulative release rates can be obtained after computation. The data of the above samples measured and obtained are as Table 2. The computation formula of the accumulative release rates is as follow:

$$Accumulative release rate = \frac{Aggregate release amount ( g )}{Aggregate amount encapsulated ( g )} \times 100 \%$$

TABLE 1
Recipe table of polymer matrix for delivery

Recipe

| | 1 | 2 |
|---|---|---|
| Chitosan acetic acid | 800 mL | 800 mL |
| solution (12.5 mg/mL) | | |
| Delivered molecule | Thmopentin | Thymopentin |
| solution (100 mg/mL) | 10 mL | 10 mL |
| Amino acid chelated | Aspartate | Glutamate |
| mineral solution (5 mM) | 200 mL | 200 mL |

TABLE 2
Analysis of the production of polymer matrix for delivery

| | Average | | Accumulative | | | |
|---|---|---|---|---|---|---|
| Sample | particle | Encapsulation | release rate (%) | | | |
| No. | size (nm) | rate (%) | 0 | 6 | 12 | 24 (hr) |
| 1 | 157 | 83 | 17 | 36 | 58 | 71 |
| 2 | 182 | 94 | 6 | 45 | 68 | 93 |
| 3 | 42 | 75 | 25 | 31 | 46 | 59 |
| 4 | 76 | 82 | 18 | 35 | 53 | 68 |
| 5 | 161 | 81 | 19 | 40 | 61 | 78 |
| 6 | 194 | 90 | 10 | 48 | 70 | 96 |
| 7 | 93 | 72 | 28 | 36 | 49 | 67 |
| 8 | 102 | 79 | 21 | 34 | 46 | 66 |
| 9 | 2370 | 21 | 79 | 91 | 99 | 99 |

The result of the above analysis of the production of polymer matrix with delivery function suggests that the particle size of the samples prepared in this invention is between 42 nm and 527 nm, and the particle size of sample No. 1˜8 is much smaller than that of the non-cross-linking (sample 9) after the polymer matrix with delivery function is ionic-bridged with amino acid and chitosan, among them the particle size of sample 3 is the smallest. There is a trend that the particle size is becoming smaller if the amount of amino acid is increased (sample No. 3, 4, 7, 8), and all their encapsulation rate is 72˜82%. Among them, sample 3, which has the accumulative release rate 59% after 24 hr of treatment, has the slowest release rate of thymopentin, and has better sustained-release ability. The result of research has not been reported in foreign and domestic literatures. From the analysis results of the two amino acid that we used in this invention, we find that using glutamate (sample No. 2, 4, 6, 8) to cross-link with chitosan can get a better encapsulation rate than using aspartate (sample No. 1, 3, 5, 7), but has worse sustained-release ability. From the analysis results of the two acid solutions that we used in this invention, using acetic acid (sample No. 1˜4) to dissolve chitosan can get better results in terms of particle size, encapsulation rate and accumulative release rates than using formic acid (sample No. 5˜8).

In summary, this invention can obtain a better encapsulation rate by employing amino acid chelated minerals solution and chitosan to proceed ionic cross-linking to prepare polymer matrix, and obtain in the same time better sustained-release ability. The means of production is simple and outstanding with stable quality and low cost, and the results can all meet our expectation. This invention is indeed an innovative technique, and so far there is no any similar invention and publication being reported. This invention should have met the requirements of a utility patent. Therefore, we, in accordance with the law, file the application for patent.

### BRIEF DESCRIPTION OF THE DRAWINGS

In order to give the esteemed patent committee better understanding on the characteristics and effects of this invention, explanation is given as follows coupled with drawings:

Drawing 1 is a preparation process of polymer matrix with delivery function

Drawing 2 is aspartate chelated minerals (pH=7 solution)

Drawing 3 is glutamate chelated minerals (pH=7 solution)

Drawing 4 is a diagram illustrating amino acid cross-link chitosan

Drawing 5 is a diagram illustrating polymer matrix with delivery function

Drawing 6 is a picture taken from the process when the polymer matrix with delivery function pass through TEM.

1) A means of production of polymer matrix for delivery, by which we firstly dissolve chitosan in acid solution to prepare chitosan acetic acid solution, and then dissolve amino acid chelated mineral and delivery molecules in water, respectively. Take adequate amount of delivery molecules and chitosan-acid solution and mix evenly, and then speedily add the amino acid chelated minerals solution into the mixed solution under the condition of high-speed homogeneity. The solution now is weak acid. Add buffer solution of pH=7, we can obtain colloidal solution of encapsulated polymer matrix with delivery function.

2) A means of claim 1 wherein the degree of acetyl group removal of chitosan is 50%˜99.9%, the average molecule weight is 1000 g/mole˜1500000 g/mole, and the concentration is 0.01˜5% (w/v).

3) A means of claim 1 wherein the acid solution employed is organic acid or inorganic acid, such as hydrochloric acid, formic acid, acetic acid, propionic acid, phosphoric acid, lactic acid, malic acid, succinic acid, nitric acid, oxalic acid, and adipic acid; its concentration is 0.1˜0.2% (w/v).

4) A means of claim 1 wherein the solution of the chitosan-acid solution is 50%˜99.9% of the colloidal volume.

5) A means of claim 1 wherein the amino acid employed is aspartate and glutamate which carry negative charge.

6) A means of claim 1 wherein the mineral metal is magnesium, calcium, strontium, zinc, copper, iron, silver, aluminum, boron and titanium ion.

7) A means of claim 1 wherein the concentration of amino acid chelated mineral solution employed is 1 mM˜1 M, the volume of the solution employed is 0.1˜50% that of the colloidal volume.

8) A means of claim 1 wherein the delivery molecule is short-chained peptide molecule, and the repeat unit number of amino acid is composed of 2˜30 molecules.

9) A means of claim 1 wherein the particle size of the colloidal solution produced is 20 mm˜500 nm.
